(19) United States
(12) Patent Application Publication
(10) Pub. No.: US 2025/0053892 A1
(43) Pub. Date: Feb. 13, 2025

(54) METHOD FOR CONTROLLING THE PRODUCTION OF A PRODUCT

(71) Applicant: BASF SE, Ludwigshafen Am Rhein (DE)

(72) Inventors: Ana ALBA PEREZ, Ludwigshafen (DE); Kurt WENZL, Ludwigshafen (DE)

(21) Appl. No.: 18/722,523

(22) PCT Filed: Dec. 19, 2022
(86) PCT No.: PCT/EP2022/086636

§ 371 Date: Jun. 20, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 10/0631; G06Q 10/087

(52) U.S. Cl. CPC G06Q 10/0631; G06Q 10/087

(57) ABSTRACT

The invention is in the field of controlling the production of a product to match a target environmental impact. It relates to a computer-implemented method for controlling the production of a product comprising
(a) receiving a request indicating the product and a target environmental impact associated with the production of the product,
(b) receiving starting material data or process-related data for the product,
(c) receiving supply material data or process-related supply data,
(d) determining, based on the request, the starting material data or process-related data and the supply material data or process-related supply data, the quantity of supply material or process-related supply associated with properties relating to an environmental impact to match the request,
(e) allocating an attribute of supply material or process-related supply to the product,
(f) generating an operational instruction related to the production of the product based on the allocation, and
(g) outputting the operational instruction.

The invention is in the field of controlling the production of a product to match a target environmental impact.

### BACKGROUND

Environmental challenges, in particular global warming and planetary boundaries, require industry to transform from conventional to production methods involving significantly reduced environmental impact. Rising customer awareness leads to an increasing demand for more products with reduced environmental impact. Such products may be those with a particularly low product carbon footprint, made from renewable raw materials, made from recycling materials, or made with renewable energy to name just a few. During the transition phase, it is required to use both conventional and starting materials and processes with reduced environmental impact in the same assets and infrastructures. These get mixed in complex production chains and yield products originating from both conventional starting materials and processes with those of reduced environmental impact. It is widely accepted that the starting materials and processes with reduced environmental impact are allocated to a certain product even if, in reality, the product is made of a mixture as long as it can be made sure that appropriate starting materials and processes with reduced environmental impact are used, and only as much of product with reduced environmental impact is sold as corresponds to the quantity of starting materials and processes with reduced environmental impact used in the production of all products. Such balancing is typically calculated by a human expert. However, this process is slow, so real-time production control is hardly possible.

### SUMMARY

The object of the invention was to provide a method to control the production of a product to match a target environmental impact of said product. The method is aimed to be flexible so it can be applied to complex production chains involving usage of the same starting material for different products as well as interconnected production chains.

These objects were achieved by the present invention. In a first aspect the invention relates to a computer-implemented method for controlling the production of a product comprising
(a) receiving a request indicating the product and a target environmental impact associated with the production of the product,
(b) receiving starting material data or process-related data for the product,
(c) receiving supply material data or process-related supply data,
(d) determining, based on the request, the starting material data or process-related data and the supply material data or process-related supply data, the quantity of supply material or process-related supply associated with properties relating to an environmental impact to match the request,
(e) allocating an attribute of supply material or process-related supply to the product,
(f) generating an operational instruction related to the production of the product based on the allocation, and
(g) outputting the operational instruction.

In another aspect the invention relates to a non-transitory computer-readable data medium storing a computer program including instructions for executing steps of the method according to the invention.

In another aspect the invention relates to a use of the operational instruction obtained by the method according to the invention for controlling the production of a product.

In another aspect the invention relates to a production control system comprising
(a) an input for
   receiving a request indicating the product and a target environmental impact associated with the production of the product,
   receiving starting material data or process-related data for the product, and
   receiving supply material data or process-related supply data,
(b) a processor for
   determining, based on the request and the starting material data or process-related data, the quantity of supply material or process-related supply associated with properties relating to an environmental impact to match the request,
   allocating an attribute of supply material or process-related supply to the product, and
   generating an operational instruction related to the production of the product based on the allocation, and
(c) an output for outputting the operational instruction.

The invention allows the fast determination of the environmental impact of a product to align it with the demand. In this way, the production can be controlled before producing the requested product, during the production or afterwards, for example by quick adjustment of the supplies for subsequent products. The control is possible even in complex production environments in which a plurality of starting materials are processed in various interconnected process steps into a plurality of products. The invention further allows a reliable control of the production and makes it easy to audit if the environmental impact of the products as labelled matches the environmental impact caused by the production in reality.

The term “controlling” as used herein generally means any direct or indirect influence on the production. An indirect influence may mean an influence on a factor related to the production, for example the dispositions for the supply delivery. Controlling also includes the verification that the claimed product characteristics are consistent with the material supply and the production processes. Such verification may trigger changes in the material supply, the production processes or the product characteristics.

The term “product” as used herein generally refers to any good which can be sold to others at any point in the value chain. This may include final products for end consumers, for example cars, paints, toys or medicaments; this may also include goods which are typically sold to other companies which further process them, for example steel parts for machines, plastic pellets for extrusion or chemical compounds, for example acrylic acid to produce superabsorbers for diapers; this may also include goods very early in the value chain like crude oil fractions, for example naphtha, agricultural products, for example soy beans, or purified sand for glass production.

The term “environmental impact” as used herein generally refers to an environmental performance of a product. The property related to the environmental impact may be associated with the environmental impact of a product at any stage during its lifecycle. The stages of the product lifecycle may include the stages of
providing raw material, components or parts to be used for producing a product,
producing products, such as intermediate products or end products,
using products,
treating end-of-life products,
recycling end-of-life products,
disposing end-of-life products,
reusing components from end-of-life products,

or any subset of stages. The property related to environmental impact may be specified or may be derived from any activity of one or more entities participating at any stage of the lifecycle of one or more product(s).

The property related to the environmental impact may include one or more characteristic(s) that are attributable to environmental impact of a product. The property related to environmental impact may include environmental, technical or circularity characteristics(s) associated with the environmental impact of one or more product(s).

Environmental characteristic(s) may specify or quantify ecological criteria associated with the products direct environmental impact. Environmental characteristic(s) may be or may be derived from measurements taken during the lifecycle of one or more product(s). Environmental characteristics may be determined at any stage of the product lifecycle and may characterize the environmental impact of the product for such stage or up to such stage. Environmental characteristic(s) may for example include product carbon footprint, greenhouse gas emissions, resource usage, air emissions, ozone depletion potential, water pollution, noise pollution or eutrophication potential.

Environmental characteristic(s) may for example include product characteristics related to the production of the product like bio based, vegan, halal, kosher, palm oil-free, natural or the like.

Technical characteristic(s) may specify or quantify product performance at least indirectly associated with the environmental impact. Technical characteristic(s) may be or may be derived from measurements taken during the lifecycle of one or more product(s). Technical characteristics may be determined at any stage of the product lifecycle and may characterize the product performance for such stage or up to such stage. Technical characteristic(s) may for example include product composition data, bill of materials, product specification data, product component data, product safety data, application property data, application instructions or product quality data. Technical characteristics may for example include data related to the quantity of cancerogenic ingredients, data related to the emission rate of volatile organic compounds, data related to the toxicity for water organisms or data related to the level of impurities.

Circularity characteristic(s) may specify or quantify the products life cycle characteristics associated with circular uses. Circularity characteristic(s) may be or may be derived from measurements taken during the lifecycle of one or more product(s). Circularity characteristic(s) may be or may be derived from circular data recorded in one or more prior lifecycle(s) including reuse. Circularity characteristics may be determined at any stage of the product lifecycle and may characterize the reuse or recycling performance for such stage or up to such stage. Circularity characteristic(s) may for example include recycling data, reuse rate, recycling rate, recycling loops, reuse reused product performance, reused product quality or the like.

According to the present invention a request for a product is received. Such request may be received through an interface, for example through an interface to a storage device, a database or an ordering system, such as a customer relation management system or an enterprise resource planning system. The request may be received directly from a customer, or it may be received from the producer of the product itself, for example a production planner, a product manager or a supply manager. The request may be received through an interface to a user interface, such as a graphical user interface for example a web interface or an app for a portable device such as a smartphone.

The request typically indicates a product, for example by its name or an identification number. The request further indicates a target environmental impact associated with the production of the product. The target environmental impact is the desired environmental impact the requested product may cause during its production taking into account starting materials and processes. The target environmental impact may contain a classifier indication the type of environmental impact and a degree of environmental impact. The degree of environmental impact may be expressed in the form of a value, a range, a maximum or minimum. It is possible that the target environmental impact contains one type of environmental impact or more than one. If it contains more than one types of environmental impacts, the target environmental impact may contain more than one classifier, wherein each classifier indicates the type of environmental impact, wherein each classifier is associated with a degree of environmental impact related to such classifier. As an example, a target environmental impact may contain the classifier product carbon footprint associated with a maximum value of 10 kg CO₂ eq per kg product and in addition the classifier recycling material content associated with a minimum value of 25%.

The request may be received before the product is produced, while the product is produced or after the product has been produced. In the latter case, the product may be stored in a warehouse or stock. In all cases the production may be controlled by adjusting the supply materials, which are either used for the requested product or are used for later produced products but allocated to the requested product. The later produced products may refer to the same kind of product or to a different one, in particular if the different one has at least one starting material or process in common with the requested product.

The term “starting material data” as used herein generally refers to a dataset related to the starting materials. Starting material generally relates to a material which is required to produce the product. If the product is produced in more than one process step, the starting material refers to all materials which have not yet been processed in a process step. This means that all materials used for the first process step are starting materials and any materials used in a subsequent step as long as they are not the output material of one of the preceding process steps. If the product is produced in more than one process step, the starting material data may comprise sub-datasets each related to one of the process steps. Starting materials may be chemical substances like benzene, silica or polyethylene. Starting materials may also be naturally grown materials like wood, starch or plant oil. Starting materials may also be components, i.e. products or parts thereof, like tires for cars, cables for electric installations in a house, or screws for furniture. Typically, starting materials are bought and shipped to a production site, where they may be kept in stock until they are used in a production process.

Starting material data usually comprises an identifier for the starting material, for example an ID number or a description. Starting material data usually also comprises the quantity of starting materials required for a product. The quantity unit can vary depending on the starting material. The quantity unit can refer to the weight, the volume, the molar amount or the number of pieces. The quantity is usually given per unit product, for example per weight, volume, molar amount or number of pieces of product.

The term “process-related data” as used herein generally refers to a dataset related to process-related items required in the process in which the product is produced from the starting materials. If the product is produced in more than one process step, the process-related data may comprise sub-datasets each related to one of the process steps. Process-related items include energy required for the production, for example electricity, heat or fuel. Process-related items further include equipment used for the production. Examples are heat exchangers which recover parts of the heat which may be used for other processes or emission capturing equipment such as carbon capturing and storing or filters removing poisonous compounds from the exhaust or from wastewater. Process-related items further include consumable material, so materials which are required for the production process but do not become part of or are converted into the product, for example lubricants for machines, sandpaper or ice for cooling. Process-related items further include transportation items, for example a container for a truck or a train and respective capacity of a truck or train. Transportation items are particularly important if the production process contains more than one process steps which are executed in different sites. According to the present invention, starting material data or process-related data are received. The starting material data or process-related data may be received from a storage medium, a database or through an interface, for example through an interface to an enterprise resource planning system. It is possible that only starting material data are received, for example if no influence on processes is possible, or that only process-related data is received, for example if no influence on the starting material is possible. More commonly, however, both starting material data and process-related data are received.

Starting material data or process-related data for a product may be readily available, in particular for simple production processes. In such cases the starting material data may be received from a database. However, for more complex production processes starting material data or process-related data may not be readily available. Complex production processes can mean that the production process contains multiple production steps each of which converts input materials into output materials, wherein at least one process step yields two output materials which are used as input material in two separate process steps or at least one process step uses output material of two separate process steps as input material. The starting materials may be used in a first step producing one or more than one output material, which are in turn used as input material for one or more subsequent steps. The production process may form a linear chain of process steps. The production process may also include branched chains of process steps, for example if an output material of one process step is used as input material in more than one subsequent process steps or if one process step yields more than one output material each of which are used in different subsequent process steps.

In such cases, data relating to each process step may be available containing the input material, the output material and process-related data for a particular process step may be received. The starting materials may hence be determined based on process step data related to the process steps required for producing the product, wherein process step data contains input material, output material and process-related data for a process step. The starting materials may be determined by starting from the last process step having the product as output material, determining any starting material in this step and identifying all process step for the remaining input materials of that step. This procedure is repeated until no preceding process step is identified anymore and hence all starting materials are determined.

The process step data may further contain a conversion factor indicating losses during a process step. For example, in a factory making furniture from wood, the cutting and sanding cause losses such that only a part of the weight of the wood starting material ends up on the product. A conversion factor of 0.8 in this example may mean that 1 kg of wood starting material is required to produce 0.8 kg of a wooden furniture. A conversion factor may in some instances be larger than 1, for example in an exothermic chemical reaction which requires heat as process-related supply to initiate the chemical reaction, but even more heat is produced by the exothermic reaction which can be reused in the same or other process steps.

The term “supply material” as used herein refers to a specific starting material associated with a property relating to an environmental impact. Hence, more than one supply material may be available for one starting material. For example, a starting material may be polyethylene-terephthalate (PET). Multiple supply materials for PET are available, for example PET produced from mineral oil, PET recycling material or bio-based PET. The supply material may be even more specific, for example by specifying the degree of bio-based material, a producer or a region it was produced.

The term “process-related supply data” as used herein refers to a dataset relating to specific process-related item used in the production process. In case of energy, the process-related data may comprise electricity as required energy source for the production process. A process-related supply may then for example be electricity from a specific gas power plant, or it may be standard electricity from the local power grid or it may be regenerative electricity, for example from a wind or solar plant. A process-related supply may also be a filter for removing dust from exhaust gas with a particular specification of the percentage of how much dust is removed from the exhaust gas.

According to the present invention the quantity of supply materials or process-related supplies is determined based on the request, the starting material data or process-related data and the supply material data or process-related supply data. The determination may involve identifying all starting materials or processes which relate to the requested environmental impact. For example, if the request contains a minimum amount of recycling materials, those starting materials need to be identified which are in principle available from recycling sources. Such determination may involve reference data containing starting materials and processes and associated classifiers for the environmental impact, wherein a starting material or a process is associated with all classifiers for the environmental impact it relates to.

For the identified starting material or process-related item, the quantity of the corresponding supply material or process-related supply is determined to match the request. As an example, the request may indicate a quota of recycling material. For the identified starting material, there may be a conventional supply material and a recycling supply material. A quantity of recycling supply material and conventional supply material is determined corresponding to the quota, thereby matching the request. There may be cases in which multiple options exist to match the request. The determination may then be based on the availability of the supply material or process-related supply, for example the option is selected for which the highest availability for the required supply material or process-related supply has been identified.

Supply material availability may be obtained from supply material data. Process-related supply availability may be obtained from process-related supply data. Supply material data or process-related supply data may be received from a storage device, a database or an interface to a management system, for example an enterprise resource management system or a supply management system. Supply material data or process-related supply data may comprise an identifier for the supply material or process-related supply and an associated attribute indicating the environmental impact of the supply material or process-related supply. The attribute may be a data object containing the classifier of environmental impact and a value indicating the degree of environmental impact. The attribute may further contain a quantity. The quantity may refer to the actual content of a container in the stock or it may be an abstract quantity, for example a certain number of kWh for electricity from renewable energy sources. The attribute may further contain a time indicator indicating a point in time or a time period, for example the time when the supply material or process-related supply was delivered. Such time indicator may be used to restrict allocation of attributes to a product only within a certain time period. Hence, the allocation may be based on the time indicator of the attribute.

More than one attribute may exist for one supply material or process-related supply. In case one supply material or process-related supply relates to more than one different classifier for the environmental impact, an attribute may exist for any of the classifiers for the environmental impact. For example, for a certain supply material an attribute may exist related to the product carbon footprint. Another attribute may exist for its content of mutagenic substances. It is also possible that for the same classifier for the environmental impact more than one attribute exists, for example to break down a large quantity into several smaller quantities in order to facilitate the allocation. Each attribute may be regarded like a currency unit which can be allocated to certain products. In addition, separate attributes may be assigned to the same supply material or process-related supply obtained from different suppliers. While the specifications may be identical or almost identical, a separation may facilitate tracking of problems related to a particular supplier which may later be detected.

The attribute may further comprise supporting data or a reference to supporting data for the environmental impact, wherein supporting data includes data evidencing that the environmental impact of the supply material or process-related supply is correctly labelled. Supporting data may involve product data sheets, certificates, audit reports or internal quality check results.

In some cases, different material supplies belonging to the same starting material may have different properties in the production process. Analogously, different process-related supplies belonging to the same process may have different properties in the production process. An example is natural gas in comparison to its analogue from bio fermentation, which may have a different caloric value due to minor compositional differences. To account for such differences, the attribute may contain a conversion unit reflecting difference of the supply material or process-related supply to a reference. The determination of the quantity of supply material or process-related supply associated with properties relating to an environmental impact may be based on the conversion unit wherein the conversion unit indicates differences of the supply material or process-related supply to a reference. The conversion unit may be a factor to be multiplied to the quantity to account for the difference of the supply material or process-related supply to a reference. The reference may, for example, be a conventional supply material or process-related supply having the conversion unit 1. Other supply material or process-related supply may have a conversion unit 0.95 indicating that the same quantity is equivalent to 0.95 times the quantity of the reference. In this case, a larger quantity needs to be determined to compensate for the difference.

According to the present invention an attribute of supply material or process-related supply is allocated to the product. The allocation is based on the determination of the quantity of supply material or process-related supply associated with properties relating to an environmental impact. The allocation may involve entering a product reference to the attribute of the required supply material or process-related supply and thereby indicating that this supply material or process-related supply in the respective quantity is not available anymore for further allocation. Alternatively, a database may be used containing the attributes and references to products to which the attributes have been allocated.

Alternatively or additionally, the allocation of the attribute of supply material or process-related supply to the product may include appending the product with associated attributes to a product attribute list and validating whether the attributes of the product attribute list do not exceed the attributes of the supply material data or the process-related data. The validation can be compared to a mass balance of the underlying products under the presumption that the mass of the supply materials and process-related supplies could be identified and quantified in the products, wherein the actual quantity in a product is irrelevant, but only the sum of all quantities is taken into account.

In some cases, in particular in larger production sites or a company controlling multiple production plants, some or all process steps may be executed in parallel in more than one production plant, for example a first process step is executed in a production plant in America and the same process step is executed in a production plant in Europe. The same process step requires the same starting materials, but different supply materials or process-related supplies associated with different environmental impacts may be available at the different production plants. It may hence not be possible that a particular supply material or process-related supply available at a first production plant contributes to a product in a second production plant, for example because the output material of the process step executed in the first production plant will never be used in a subsequent process step of the second production plant. It may be desirable to make sure that the allocation of an attribute of supply material or process-related supply to the product is restricted to the actual material flow. Hence, the allocation may include validating whether the supply material or process-related supply to be attributed is part of a material exchange chain to the requested product. For this purpose, the supply material data or the process-related supply data may include the indication of the production plant in which the supply material or process-related supply is used.

According to the present invention, an operational instruction is generated based on the allocation. The term “operational instruction” as used herein generally refers to any data which can be used to influence the production process by any means. The operational instruction may contain the confirmation that the requested product with the target environmental impact can be shipped to the customer. The operational instruction may, hence, either trigger the production process of the request product or it may trigger the shipment of the product to the customer if the product is already in stock. The operational instruction may also indicate that the requested product cannot be shipped with the target environmental impact. In this case, the consequence can be that only a lower quantity of the product can be shipped to the customer or that respective supply materials or product-related supplies need to be acquired in order to match the target environmental impact.

According to the present invention the operational instruction is output. The operational instruction can be output to a data storage medium or a database. The operational instruction can also be output through an interface, for example through an interface to a user interface or a customer relation management system or an enterprise resource planning system. The user interface may be a graphical user interface for example a web interface or an app for a portable device such as a smartphone. The operational instruction may be output through a user interface to a production planner, a product manager or a supply manager. The operational instruction may be output to the same system or interface from which the request for the product is received or from a different one. The operational instruction may be output to one system or interface or to more than one.

In another aspect, the invention relates to non-transitory computer-readable data medium storing a computer program including instructions for executing steps of the method of the present invention. “Computer-readable data medium” refers to any suitable data storage device or computer readable memory on which is stored one or more sets of instructions (for example software) embodying any one or more of the methodologies or functions described herein. The instructions may also reside, completely or at least partially, within the main memory and/or within the processor during execution thereof by the computer, main memory, and processing device, which may constitute computer-readable storage media. The instructions may further be transmitted or received over a network via a network interface device. Computer-readable data medium include hard drives, for example on a server, USB storage device, CD, DVD or Blue-ray discs. The computer program may contain all functionalities and data required for execution of the method according to the present invention or it may provide interfaces to have parts of the method processed on remote systems, for example on a cloud system. The term “non-transitory” has the meaning that the purpose of the data storage medium is to store the computer program permanently, in particular without requiring permanent power supply.

In another aspect, the invention relates to use of the operational instruction obtained by the method according to any one of the preceding claims for controlling the production of a product. The operational instruction can be used to initiate the production process of the requested product. The operational instruction can be used to confirm the shipment of a product with the target environmental impact to the customer. It can also be used to change the quantity of the requested product. The operational instruction can be used to trigger the ordering of supply material or process-related supply with particular requirements for the environmental impact associated with the supply material or process-related supply.

In another aspect, the invention relates to production control system. The production control system is configured for executing the method of the present invention. Hence, the description and preferred embodiments described for the method above also applies to the production control system. The production control system may be a standalone system, or it may be part of or a module in an enterprise resource planning system or a customer relation management system.

The production control system has an input for receiving a request and starting material data or process-related data. The input can be a data connection a storage device or a database, for example a wired connection or a telecommunication connection. The input can also be or contain an interface to a

### BRIEF DESCRIPTION OF THE FIGURES

FIG. 1 depicts an implementation of the method of the invention.

FIG. 2 depicts an option to determine the starting materials of a product from process step data.

FIG. 3 depicts an implementation of the system of the invention.

FIG. 4 depicts an example of a complex production process.

FIG. 5 depicts an example of the method for the production process of FIG. 4.

FIG. 6 depicts further details of the example of the production process of FIG. 4.

FIG. 7 depicts an example for a complex production process distributed in two different production sites.

FIG. 8 depicts another example for a production process.

FIG. 9 depicts further details of the example of FIG. 8.

FIGS. 10, 11 and 12 depict examples for user interfaces for a request.

### DESCRIPTION OF EMBODIMENTS

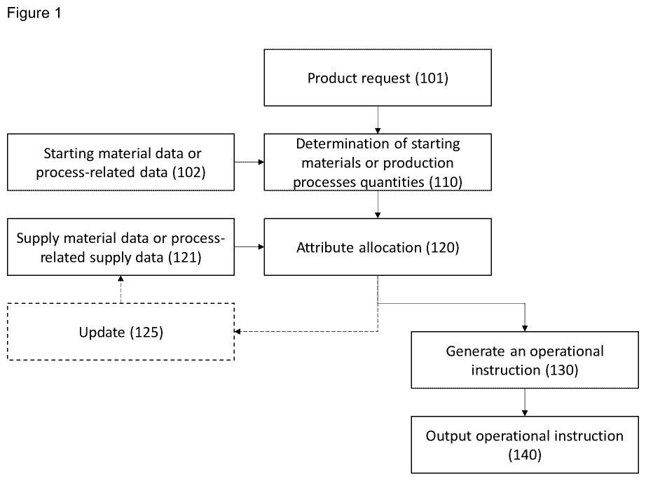

In FIG. 1 an implementation of the method of the invention is depicted. A product request (101) is received, for example from a web application or an interface to a customer relation management system. The product request (101) indicates the product, for example the name of a product or a product reference such as an order number for the product. The product request (101) further indicates a target environmental impact associated with the production of the product. The target environmental impact can relate to a product carbon footprint, for example a maximum product carbon footprint the requested product may have or a range of product carbon footprints. The target environmental impact can also relate to a content of a specific harmful compound, for example formaldehyde or bisphenol-A. The target environmental impact can also relate to the energy used in the production process, for example only renewable energy like solar or wind energy is used for the production of the product.

Starting material data or process-related data (102) are also received, for example from a storage device or an interface to an enterprise resource planning system. Starting material data typically contains the materials which are purchased for the production. Starting material data may contain the name of or a number referring to a starting material, its required quantity and sometimes also the quality. An example for a starting material may be 20 oak wood boards having a thickness of 25 mm and a width of 200 mm and a length of 2 m. Process-related data refers to a process-related item required in the process in which the product is produced from the starting materials, for example a piece of equipment, energy, or a consumable material. Process-related data may contain the name of or a reference to a process-related item, its quantity and sometimes the quality. For example, process-related data may contain the information that 21 kWh of electricity are required as well as 0.3 m³ of cooling water at a temperature of or below 20° C.

The product request (101) and the starting material data or process-related data (102) are used to determine the starting materials or production processes quantities (110). In some cases, this can be a simple multiplication, for example a product request (101) relates to 100 kg of a product and the starting material data or process-related data (102) contain a list of starting materials and process-related items per 1 kg of this product, the list may be multiplied by the factor 100 to arrive at the starting materials or production processes quantities.

Supply material data or process-related supply data (121) is received. Supply material data contains data related to the supply material, i.e. specific starting material associated with a property relating to an environmental impact. process-related supply data contains data relates to specific process-related items used in the production process. Supply material data or process-related supply data (121) contains the name of or a reference to the supply material or process-related supply and an associated attribute indicating the environmental impact of the supply material or process-related supply.

The attribute of supply material or process-related supply is allocated to the product (120). Such allocation indicates that the quantity of the determined supply material data or process-related supply are associated with the requested product and may hence not be associated any more with a different product. In a way, the attributed in the respective quantities gets consumed by the allocation. The allocation is independent of the fact if the respective supply materials are in reality used to produce the product or not. In particular in larger production sites different supply materials and process-related supplies get mixed in large apparatuses, so in reality it is impossible to determine if and to what extent a supply materials or process-related supplies physically ended up in the product. The allocation is stored to avoid any double usage of the attributes allocated a certain product. This can be achieved in various ways, for example in an allocation database. It is also possible to add a flag to the attribute itself. In this case the allocation may involve or be followed by an update (125) of the supply material data or process-related supply data (121).

Based on the allocation an operational instruction is generated (130). The operational instruction may contain the confirmation that the requested product can be produced and/or be shipped to the customer in case the allocation was successful. If no allocation was possible, for example because insufficient attributes associated with required supply material or process-related supply were available, the operational instruction may contain a trigger to acquire the lacking supply material or process-related supply. The operational instruction is output (140), for example through an interface to an enterprise resource planning system or a user interface. In this way, the operational instruction controls the production, either directly by triggering direct action or indirectly by pointing operators to take the required action.

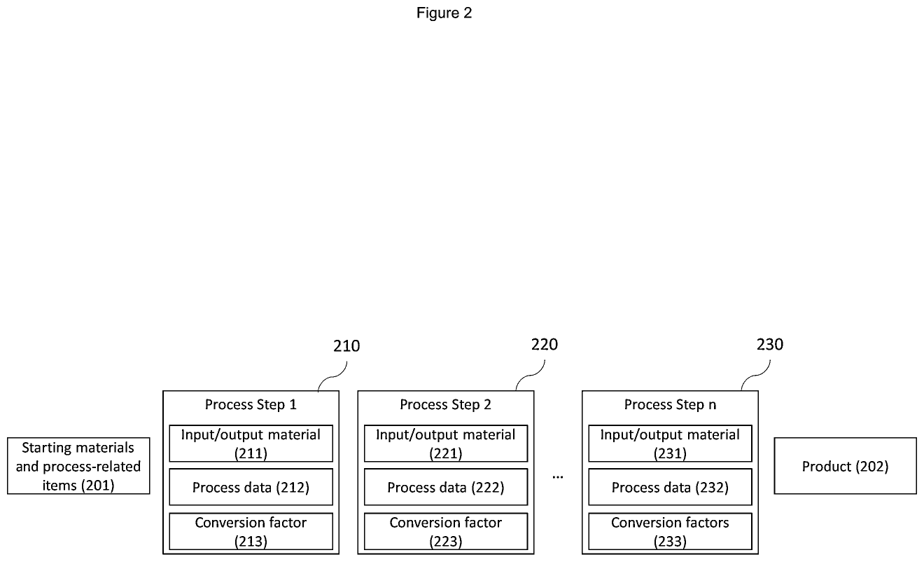

FIG. 2 illustrates a way of determining the starting materials and process-related items (201) for a product (202) for the case that the product (202) is produced in multiple process steps from starting materials and process-related items (201). In complex production processes, such data may not be readily available, but it has to be determined based on information about each process step. Each process step (210, 220, 230) may contain data about its respective input and output materials (211, 221, 231), its processes (212, 222, 232) and sometimes also conversion factors (213, 223, 233). Starting from a product, a process step n may be determined having the product as output material. All input materials of process step n which are output materials of other process step indicate the previous process steps. All input materials of process step n which are not output materials of any other process step qualify as starting material. Repeating this analysis for all previous process steps until no previous process step can be found any more provides all involved process steps and all starting materials. The process-related items can be retrieved from the process step data (210, 220, 230). The conversion factors may indicate the portion of the input materials and process-related items for an output material of interest. This is particularly relevant if a process step yields two or more output materials out of which only one is required for producing the product. In this case, for example, for one unit of an output material only half of the required input materials are calculated for this output material and the other half is calculated for the other output material. The conversion factor can also reflect other deviations, for example losses due to limited yields, purifications or packaging.

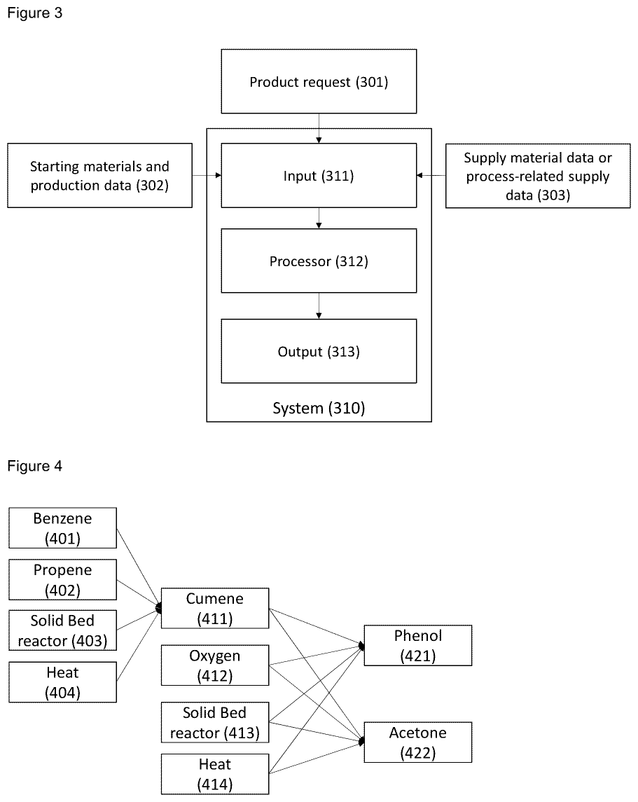

FIG. 3 illustrates an embodiment for a production control system (310) for executing the method of the invention. The system (310) contains an input (311) to receive the product request (301), starting material or process-related data (302), and supply material data or process-related supply data (303). The input (310) may be a connection to a storage device or an interface to a database or a user interface. The input (310) passes the received data to a processor (312). The processor (312) is adapted to determining, based on the request and the starting material data or process-related data, the quantity of supply material or process-related supply associated with properties relating to an environmental impact to match the request, allocating an attribute of supply material or process-related supply to the product, and generating an operational instruction related to the production of the product based on the allocation. The operational instruction is passed to an output (313) which outputs the operational instruction. The output may be a connection to a storage device or an interface to a database or a user interface. The production control system (310) may be a single computing device or multiple connected computer systems. For example, the input (311) may be on one computer device, the processor (312) may be in a could system and the output (313) may be on another computer device.

FIG. 4 depicts an example of a complex production process in a chemical plant. The production process contains two process steps. In a first step, benzene (401) and propene (402) are reacted in a solid bed reactor (403) using heat (404), for example from a steam pipeline, to form cumene (411). This cumene (411) is reacted in a second step with oxygen (412) in another solid bed reactor (413) using again heat (414) to form two products, namely phenol (421) and acetone (422).

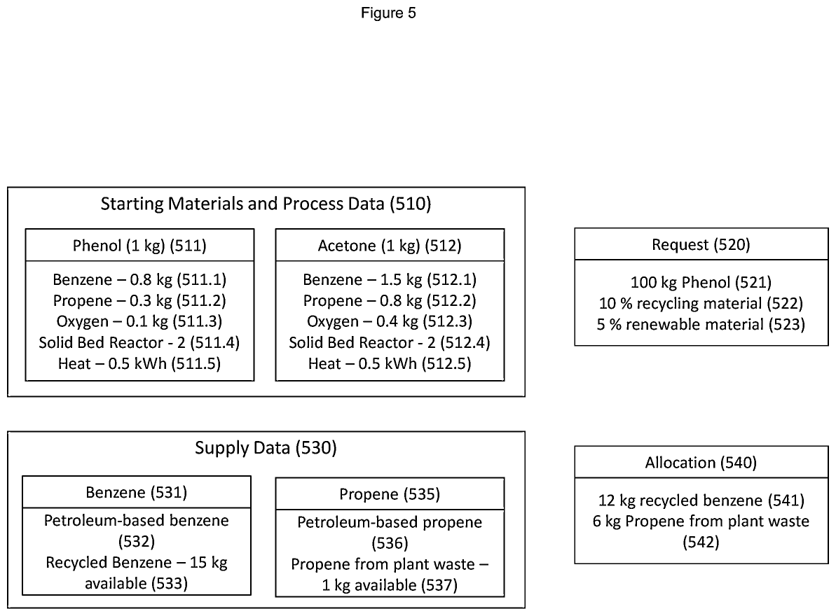

FIG. 5 depicts the involved data for the example of FIG. 4. The starting materials and process-related data (510) may contain two data subsets, one for phenol (511) and one for acetone (512). Both data subsets relate to one kilogram of phenol (511) or acetone (512). The data subset for phenol (511) contains a quantity of 0.8 kg benzene (511.1), 0.3 kg propene (511.2), 0.1 kg oxygen (511.3), two solid bed reactor (511.4) and 0.5 kWh heat (511.5). The data subset for acetone (512) contains a quantity of 1.5 kg benzene (512.1), 0.8 kg propene (512.2), 0.4 kg oxygen (512.3), two solid bed reactors (512.4) and 0.5 kWh heat (512.5).

A request (521) may contain the request for 100 kg phenol (521), wherein the phenol is made of 10% recycling material (522) and 5% renewable material (523).

Supply data (530) may contain two data subsets, one for benzene (532) and one for propene (533). The data subset for benzene (532) contains petroleum-based benzene (532) and recycled benzene (533). The latter is associated with an attribute indicating that 15 kg of the recycled benzene is available. The data subset for propene (535) contains petroleum-based propene (536) and propene from plant waste (537). The latter is associated with an attribute indicating that 1 kg is available.

Based on the starting materials and process-related data (510), the request (521) and the supply data (530) an allocation (540) which matches the degree of environmental impact in the request may be effected. The allocation (540) involves the attributes related to 12 kg recycled benzene (541) and 6 kg propene from plant waste (542).

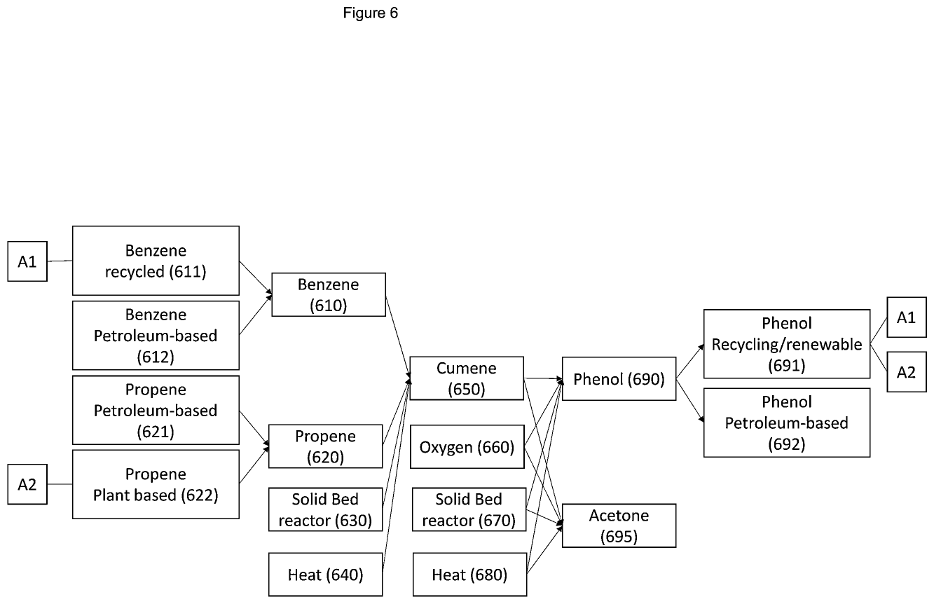

FIG. 6 illustrates the embedding of the attributes of the supply materials and the product into the complex production process. Benzene (610) is used from two supply materials, namely recycled benzene (611) and petroleum-based benzene (612). Recycled benzene (611) is associated with attribute (A1) reflecting the reduced environmental impact due to the recycling source. The two benzene sources get mixed in the process, so all obtained products are physically each based partially on recycled benzene (611) and petroleum-based benzene (612). However, the attribute (A1) may be allocated to either phenol or acetone or both, as long as the quantities for the attribute A1 on the product side is equal or less than the quantities of the attribute on the supply materials side. In this example, the attribute A1 is fully allocated to phenol in order to match the product request requesting phenol with such an attribute. This product is therefore referred to as phenol from recycling material (691), while the remaining phenol is conventional phenol (692) or petroleum-based phenol. As the attribute A1 is fully used for phenol (691), acetone (695) can only be obtained as conventional, namely petroleum-based, unless more recycled benzene (611) is acquired.

Equivalently, propene (620) is used from two supply materials, namely petroleum-based (621) and plant-based (622), which means that it has been obtained by pyrolysis of plant materials which can regrow. Plant-based propene (622) is hence associated with attribute (A2) indicating the renewable source. Propene from the two sources gets mixed in the production process to form phenol (690) and acetone (695). To match the request for phenol from renewable resources, the attribute is allocated to phenol to obtain phenol from renewable resources (691). The remaining phenol is obtained as conventional or petroleum-based phenol (692). Once again, attribute A2 is used up for phenol, so acetone is only obtained as conventional or petroleum-based acetone (695), unless more propene from renewable sources (622) is acquired.

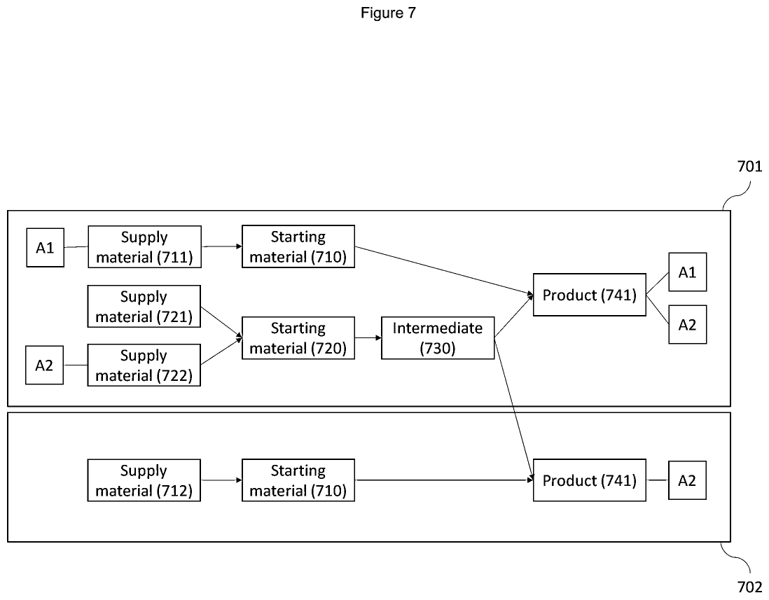

FIG. 7 depicts an example for a complex production process distributed over two different production sites. In the first production site (701), which may be located in Europe, starting material (720) is converted into intermediate (730). This intermediate (740) is converted together with starting material (710) into product (741). In a second production site (702), which may be located in North America, the same product (741) is produced in the same way, wherein intermediate (730) is obtained from the first production site (702), i.e. the intermediate (730) is shipped from the first production site (701) to the second production site (702). The starting material (710) is not obtained from the first production site (701), but acquired from a separate source.

As starting material (710) in the first production site (701) a supply material (711) is used which has an associated attribute (A1) relating to a particular environmental impact. In the second production site (702) for the same starting material (710) a different supply material (712) is used. This does not have an attribute (A1), for example because it is a conventional supply material. Production site (701) uses for starting material (720) a mixture of two supply materials, namely supply material (721) and supply material (722). The latter is associated with attribute (A2). Product (741) obtained in production site (701) may be attributed to both attributes (A1) and (A2) as both supply materials may physically end up in product (741). However, product (741) of production site (702) may only be allocated to attribute (A2) when taking into account the fact that all product in that production site will always be made from conventional supply material (721), i.e. there is no way that at least some of supply material (711) physically ends up in product (741) in production site (702). Consequently, attribute A1 may in this case not be attributed to product (741) of production site (702) even if sufficient supply material (711) is available at production site (701).

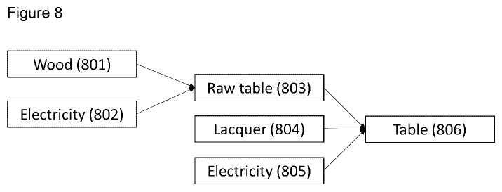

FIG. 8 illustrates another example for the method of the invention. This example illustrates the method for piece-good, such as pieces of furniture like a table. The table (804) is produced in two process steps, which is largely simplified for the sake of illustrating the invention. In reality, a lot more process steps are involved. For the present purpose, in a first process step, the starting material wood (801) and the process-related item electricity (802) are processed into a raw table (803), i.e. a table without any surface finish. In a second process step, the raw table (803) is treated with a lacquer (804) as another starting material and again electricity (805) as process-related item for applying the lacquer, for example with a spray robot. The second process step yields a table (806) as the product.

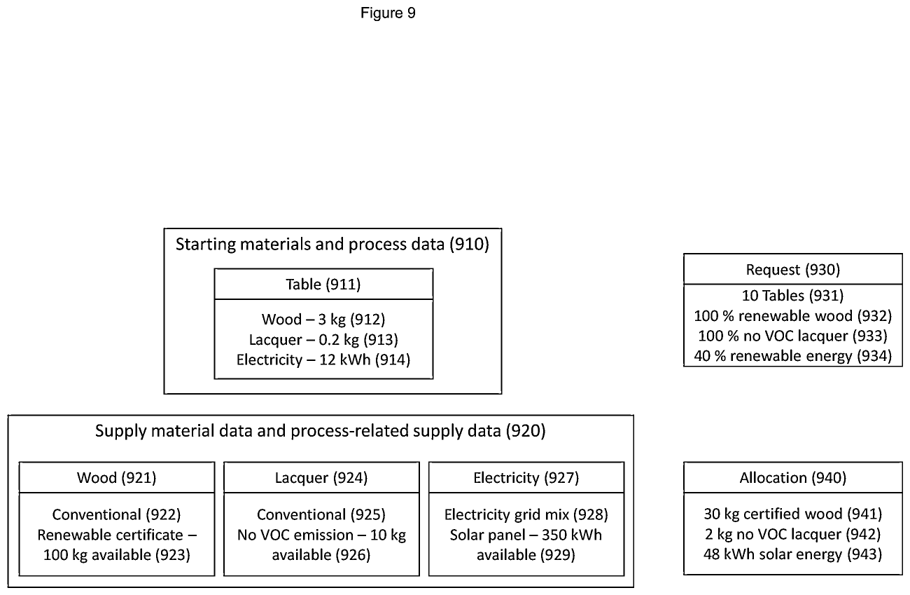

FIG. 9 depicts the datasets relating to the example of FIG. 8. The starting materials and process data (910) contains a dataset for a table (911), wherein this dataset contains all starting material together with their quantities required to produce one table. It contains 3 kg wood (912) 0.2 kg lacquer (913) and 12 kWh electricity (914). The supply material data and process-related supply data (920) contains three data subsets, one each for wood (921), lacquer (924) and electricity (927). The data subset for wood (921) contains conventional wood (922) as well as wood from certified renewable foresting (923). The certified wood (923) is associated with an attribute indicating that 100 kg of it were not yet allocated to any product and are hence available for allocation. The certified wood (923) may also be associated with the certificate, for example the data subset (921) contains a reference to this certificate or it contains the entire content of the certificate. The data subset for lacquer (924) contains conventional lacquer (925) as well as lacquer having not volatile organic compound (VOC) emission (926). The no VOC lacquer (926) is associated with an attribute indicating that 10 kg of it were not yet allocated to any product and are hence available for allocation. The data subset for electricity (927) contains electricity obtained from the electricity grid mix (928) and electricity for a solar panel (929). The solar panel electricity (929) is associated with an attribute indicating that 350 kWh of it were not yet allocated to any product and are hence available for allocation.

A request (930) indicates a request for 10 tables (931), wherein the tables are targeted to be made from 100% renewable wood (932), 100% lacquer emitting no VOC (933) and 40% renewable energy (934). Accordingly, an allocation (940) may include allocating 30 kg certified wood (941), 2 kg no VOC lacquer (942) and 48 kWh solar energy (943) to the requested tables.

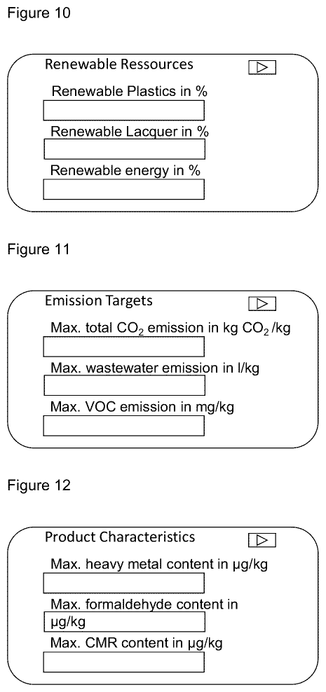

FIGS. 10, 11, and 12 depict an example for a user interface for receiving the request for a product together with the targeted environmental impact of the product. The user interface may collect input data related to the renewable resources the product is desired to be made from as shown in FIG. 10. As an example, the content of renewable plastics in %, the content of renewable lacquer and the content of renewable energy is collected as user input. The user interface may further collect data related to the emission targets as shown in FIG. 11. As an example, the maximum total CO₂ emission in kg CO₂/kg, the maximum wastewater emission in I/kg and the maximum VOC emission in mg/kg is collected as user input. The user interface may further collect data related to the product characteristics as shown in FIG. 12. As an example, the maximum heavy metal content in μg/kg, the maximum formaldehyde content in μg/kg and the maximum content of cancerogenic, mutagenic and reproduction toxic (CMR) compounds in μg/kg. After having collected such input data, the user interface may provide this data together with the requested product to the interface to the production control system of the invention.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality. A single unit or device may fulfill the functions of several items recited in the claims. The mere fact that certain measures are recited in mutually different dependent claims does not indicate that a combination of these measures cannot be used to advantage.

1. A computer-implemented method for controlling the production of a product comprising
(a) receiving a request indicating the product and a target environmental impact associated with the production of the product,
(b) receiving starting material data or process-related data for the product,
(c) receiving supply material data or process-related supply data,
(d) determining, based on the request, the starting material data or process-related data and the supply material data or process-related supply data, the quantity of supply material or process-related supply associated with properties relating to an environmental impact to match the request,
(e) allocating an attribute of supply material or process-related supply to the product,
(f) generating an operational instruction related to the production of the product based on the allocation, and
(g) outputting the operational instruction.

2. The computer implemented method according to claim 1, wherein the target environmental impact contains a classifier indication the type of environmental impact and a degree of environmental impact.

3. The computer implemented method according to claim 2, wherein the target environmental impact contains more than one classifier, wherein each classifier indicates the type of environmental impact.

4. The computer implemented method according to claim 1, wherein the environmental impact relates to the product carbon footprint or the recycling rate.

5. The computer implemented method according to claim 1, wherein the product is produced in more than one process step and wherein the starting material data or the process-related data comprises sub-datasets each related to one of the process steps.

6. The computer implemented method according to claim 1, wherein the process-related data includes data related to the energy required for the production.

7. The computer implemented method according to claim 1, wherein the production contains multiple production steps each of which convert input materials into output materials, wherein at least one process step yields two output materials which are used as input material in two separate process steps or at least one process step uses output material of two separate process steps as input material.

8. The computer implemented method according to claim 1, wherein the determination of the quantity of supply material or process-related supply associated with properties relating to an environmental impact is based on a conversion unit wherein the conversion unit indicates differences of the supply material or process-related supply to a reference.

9. The computer implemented method according to claim 1, wherein the allocation includes validating whether the supply material or process-related supply to be attributed is part of a material exchange chain to the requested product.

10. The computer implemented method according to claim 1, wherein the attribute further contains a time indicator and wherein the allocation is based on the time indicator of the attribute.

11. The computer implemented method according to claim 1, wherein the attribute further comprises supporting data or a reference to supporting data for the environmental impact, wherein supporting data includes data evidencing that the environmental impact of the supply material or process-related supply is correctly labelled.

12. A non-transitory computer-readable data medium storing a computer program including instructions for executing steps of the method according to claim 1.

13. A method for controlling the production of a product comprising using the operational instruction obtained by the method of claim 1.

14. A production control system comprising
(a) an input for
receiving a request indicating a product and a target environmental impact associated with the production of the product,
receiving starting material data or process-related data for the product, and
receiving supply material data or process-related supply data,
(b) a processor for
determining, based on the request and the starting material data or process-related data, the quantity of supply material or process-related supply associated with properties relating to an environmental impact to match the request,
allocating an attribute of supply material or process-related supply to the product, and
generating an operational instruction related to the production of the product based on the allocation, and
(c) an output for outputting the operational instruction.

15. The production control system according to claim 14, wherein the input is a graphical user interface.
